(19) United States
(12) Patent Application Publication
(10) Pub. No.: US 2005/0136448 A1
(43) Pub. Date: Jun. 23, 2005

(54) Apparatus and methods for fluorescent detection of nucleic acids

(75) Inventors: Kirk D. Hartel, West Fargo, ND (US); Mark J. Pavicic, Fargo, ND (US); Gregory Gillispie, Fargo, ND (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(73) Assignee: Dakota Technologies, Inc., Fargo, ND (US)

(21) Appl. No.: 10/954,942

(22) Filed: Sep. 29, 2004

Related U.S. Application Data
(60) Provisional application No. 60/508,556, filed on Oct. 2, 2003.

(51) Int. Cl. C12Q 1/68; C12M 1/34; G03C 5/16

(52) U.S. Cl. 435/6; 435/287.2; 250/581

(57) ABSTRACT

Apparatus and methods are provided to monitor hybridization of a probe polynucleotide for detection and analysis of a target polynucleotide in a test sample. A single-stranded probe polynucleotide is attached to an identifiable region on a substrate and allowed to hybridize to a complementary target polynucleotide that may be present in a test sample. A fluorophore noncovalently interacts with the double-stranded polynucleotides that may be formed. The fluorescence decay or lifetime of the fluorophore associated with the double-stranded polynucleotide is different from the fluorescence decay or lifetime of the fluorophore when it is associated with a single-stranded polynucleotide. The detection of double-stranded polynucleotides at a particular region is an indication that the target polynucleotide is present in the test sample.

### TECHNICAL FIELD

This invention relates to apparatus and methods to detect hybridization of a probe polynucleotide for detection of a target polynucleotide in a test sample. The apparatus includes a pulsed light source and a digitizer.

### BACKGROUND OF THE INVENTION

Nucleic acid detection methods are widely utilized in research and development, drug discovery, biodefense, and diagnostic applications. A polynucleotide probe (single-stranded polynucleotide that is complementary to a specific target polynucleotide) may be used to selectively identify the presence of a particular target polynucleotide via hybridization. Fluorescence is widely utilized because of its high degree of sensitivity to detect these hybridization events. [003] One traditional method for fluorescence-based polynucleotide detection relies on amplification of a target polynucleotide by the polymerase chain reaction (PCR) and the incorporation of a fluorophore label. Although PCR is sensitive, the cost is relatively high, it has limited multiplex potential, requires numerous reagents, and the process typically takes more than 30 minutes. Other methods rely on covalently-labeled probes that exhibit a change in fluorescence intensity upon hybridization to a polynucleotide target. However, covalent labeling of probes with fluorophores is relatively expensive and may be subject to complicated and highly restrictive design rules.

Alternatively, all of the polynucleotides in a test sample may be covalently labeled with a fluorophore and allowed to hybridize to probe polynucleotides attached to known locations on a surface. The fluorescence signal is used to delineate the location of the fluorophore-labeled target polynucleotide on a solid surface and thus identify the presence of a specific target polynucleotide. This method is complex, expensive, and not suitable for analysis of a large number of test samples.

Certain fluorophores can be used to stain polynucleotides without the need for the fluorophore to be covalently linked to either the target or probe polynucleotide. These polynucleotide-staining dyes are inexpensive, easy to use, and can provide ultra-sensitive detection of polynucleotides. However, these dyes lack specificity for a target polynucleotide (will bind to nonspecific polynucleotides), and they will generally interact to a certain degree with both single-stranded and double-stranded polynucleotides. Binding of the dye to the single-stranded probe polynucleotides and the single-stranded regions flanking the hybridization domain of the target polynucleotide will confound an intensity-based measurement to monitor hybridization.

This invention was made to allow the use of polynucleotide staining dyes for multiplex, target-specific detection of polynucleotides. By reducing the number of reagents and eliminating the need for covalent attachment of fluorophores to probe or target polynucleotides, this invention provides tremendous advantages in speed, cost, simplicity, and sensitivity over currently available technologies.

### SUMMARY OF THE INVENTION

Apparatus and methods are provided for detecting and quantitating hybridization between a single-stranded probe polynucleotide attached to a substrate and a target polynucleotide. Hybridization is detected by measuring the fluorescence decay and/or lifetime of a fluorophore noncovalently bound to the polynucleotides, wherein the fluorescence decay of the fluorophore noncovalently associated with a double-stranded polynucleotide is different from the fluorescence decay of the fluorophore if it is noncovalently associated with a single-stranded polynucleotide. Suitable fluorophores for this invention include, but are not limited to, SYBR Green I and PicoGreen (Molecular Probe Inc., Eugene Oreg.).

The apparatus includes a substrate wherein a probe polynucleotide is attached to an identifiable region of the substrate. The probe is substantially complementary, e.g., complementary to a hybridization domain within a target polynucleotide that may or may not be present in a test sample. Preferably, the substrate contains more than one identifiable region where each identifiable region contains a different probe polynucleotide to allow for multiplex analysis of different target polynucleotides in a test sample.

Alternatively, the same probe polynucleotide can be attached to a multiplicity of identifiable regions to assay a multiplicity of test samples for a target polynucleotide.

In some instances, a multiplicity of probe polynucleotides are attached at one of the identifiable regions. If a target polynucleotide hybridizes to such a region, the test sample may be assayed with a different substrate containing each of the multiple probe polynucleotides separately attached to different identifiable regions of the substrate. This provides for the identification of the probe polynucleotide(s) that originally hybridized to the target polynucleotide.

The substrate can be in any format and configuration. It can be a bead array, encoded particle array, a traditional microarray, membrane, or a microwell plate.

The apparatus also includes a fluorescence decay detection system capable of measuring the fluorescence decay and lifetime of a fluorophore at each region. The fluorescence decay detection system comprises a pulsed light source and a digitizer. The pulsed light source can be a microlaser, preferably a solid-state passively q-switched laser that can produce laser pulses with short time intervals of duration (e.g., in the sub-nanosecond or nanosecond, such as 0.4 ns to several nanosecond range). A particularly preferred digitizer is a transient digitizer that can be used to sample fluorescent signals at about a 0.5 gigahertz or higher sampling rate.

In the methods of the invention, a fluorescently labeled polynucleotide hybridization complex is formed. The complex contains a probe polynucleotide attached to a substrate, a target polynucleotide (if present in a test sample) and a fluorophore that noncovalently interacts with at least the double-stranded region of the complex. The fluorescence decay or lifetime of the fluorophore is measured to provide an indication of the presence or absence of the target polynucleotide in the test sample. The fluorophore is chosen so that it has a different fluorescence decay or lifetime when noncovalently associated with double-stranded polynucleotide as compared to if and when it is noncovalently associated with a single-stranded polynucleotide.

The fluorescently labeled hybridization complex is typically formed by contacting a test sample with one or more probe polynucleotides attached to a substrate where the probe polynucleotide hybridizes to a hybridization domain within a target polynucleotide. The contacting is under conditions that permit hybridization between the probe polynucleotide and the hybridization domain in the target polynucleotide, if present, in the test sample. The fluorescently labeled hybridization complex also contains a fluorophore that noncovalently binds to the double-stranded region of the hybridization complex. The fluorophore may be added before hybridization (e.g., with the immobilized probe polynucleotide or the target polynucleotide) or during or after the formation of the hybridization complex. The fluorescence decay and/or lifetime of the fluorophore is measured to provide an indication of whether the target polynucleotide is present in the test sample at the identifiable region of the substrate. The fluorescence decay and/or lifetime of the fluorophore when associated with a double-stranded polynucleotide is different from the fluorescence decay or lifetime if the fluorophore is associated with a single-stranded polynucleotide. In a preferred embodiment, the substrate has identifiable regions in which the probe polynucleotides are attached.

The fluorescence decay and/or lifetime of the fluorophore can be measured using any appropriate fluorescence detector and measurement techniques. However, particularly preferred fluorescence detection systems are those described herein in connection with the apparatus of the invention.

Other features, objects, and advantages of the present invention are apparent in the detailed description that follows. It should be understood, however, that the detailed description, while indicating preferred embodiments of the invention, are given by way of illustration only, not limitation. Various changes and modifications within the spirit and scope of the invention will become apparent to those skilled in the art from the detailed description.

### BRIEF DESCRIPTION OF THE DRAWINGS

FIG. 1 is a schematic diagram showing single-stranded probe polynucleotides attached to a surface.

FIG. 2 is a schematic illustration of the complementary region of a target polynucleotide hybridizing to a probe polynucleotide while a non-target polynucleotide remains unbound.

FIG. 3 illustrates the fluorescent staining of target and probe polynucleotides after hybridization without evacuation of the fluorescent staining solution.

FIG. 4 illustrates a digitizer that may be used in the present invention.

FIG. 5 is a block diagram of the architecture for a digitizer with analog memory and a DSP in accordance with one embodiment.

FIG. 6 is a schematic diagram of the sample signal capture and data flow is a system according to one embodiment.

FIG. 7 is a schematic block diagram of another embodiment.

FIG. 8 is a timing diagram showing the relative time scales for sample capture and subsequent signal processing for two fluorescence decay waveforms.

FIG. 9 displays the normalized fluorescence decay waveforms for SYBR Green I bound to ssDNA and dsDNA in solution.

FIG. 10 displays the normalized fluorescence decay waveforms of SYBR Green I for samples with varying ratios of dsDNA and ssDNA in solution.

FIG. 11 displays the differences in SYBR Green I waveforms (denoted by chi-squared values) between samples with varying ratios of dsDNA and ssDNA in solution.

### DETAILED DESCRIPTION

This invention provides apparatus and methods to detect and/or quantitate the hybridization of a probe polynucleotide with a target polynucleotide in a test sample. The apparatus uses a single-stranded probe polynucleotide(s) attached to an identifiable region(s) on a substrate, wherein the probe polynucleotide(s) is (are) substantially complementary to a hybridization domain in one or more target polynucleotide of interest. The substrate may contain a multiplicity of identifiable regions where for example: (1) different probe polynucleotides are attached to each region to allow multiplex analysis of different target polynucleotides; (2) the same probe polynucleotides are used to assay a multiplicity of test samples, and/or (3) a multiplicity of different probe polynucleotides are attached to one or more individual identifiable regions. One or more of the regions may also contain a fluorophore that noncovalently interact with double-stranded polynucleotides. Suitable fluorophores have a different fluorescence decay and/or lifetime when associated to double-stranded polynucleotides as compared to single-stranded polynucleotides.

One or more of the identifiable regions are contacted with one or more test samples under conditions that allow hybridization to occur between the probe polynucleotide(s) and the hybridization domain(s) of the target polynucleotide(s) that may or may not be present in a test sample. After hybridization, the test sample may be removed from the identifiable regions (e.g. by washing). This is to remove polynucleotides that do not hybridize to the probe polynucleotides.

The fluorophore is allowed to noncovalently bind with the probe polynucleotide(s), the target polynucleotide(s), and/or the hybridization complexes that may be formed. The fluorescence decay and/or lifetime of the fluorophore associated with the double-stranded polynucleotide complex is different from the fluorescence decay and/or lifetime of the fluorophore if it is associated with a single-stranded polynucleotide. Accordingly, if a fluorophore binds to single-stranded polynucleotides (e.g., outside the hybridization domain of the target polynucleotide or with single-stranded probe polynucleotide) it can be distinguished from the fluorescence decay or lifetime of the fluorophore bound to the double-stranded region of the hybridization complex. This avoids the significant difficulty associated with the use of fluorescence intensity to detect hybridization. The decay or lifetime of the bound fluorophore is independent of the intensity of the signal.

The fluorescence decay and/or fluorescence lifetime of the fluorophore within said regions may also then be used to detect and quantitate hybridization between the probe polynucleotide(s) and target polynucleotide(s) as a measure of the amount of target in the test sample.

By “polynucleotide,” “nucleic acid,” “oligonucleotide” or grammatical equivalents herein means at least two nucleotides covalently linked together. A polynucleotide of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of polynucleotide (see Jenkins et al., Chem. Soc. Rev. (1995) pp169-176). Several polynucleotide analogs are described in Rawls, C & E News Jun. 2, 1997 page 35. All of these references are hereby expressly incorporated by reference. These modifications of the ribose-phosphate backbone may be done to increase the stability and half-life.

Peptide nucleic acids (PNA) include peptide nucleic acid analogs. These backbones are substantially non-ionic under neutral conditions, in contrast to the highly charged phosphodiester backbone of naturally occurring polynucleotides. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The polynucleotides may be single-stranded or double-stranded, as specified, or contain portions of both double-stranded or single-stranded sequence. The polynucleotide may be DNA, both genomic and cDNA, RNA or a hybrid, where the polynucleotide contains any combination of deoxyribo- and ribo-nucleotides, and any combination of bases, including uracil, adenine, thymine, cytosine, guanine, inosine, xathanine hypoxathanine, isocytosine, isoguanine, etc.

As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

“Probe polynucleotide” or “probe” herein may be any of the aforementioned polynucleotides. Probe polynucleotides are designed to have a region that has a nucleotide sequence (the probe hybridization domain) that is complementary to a hybridization domain in a target polynucleotide such that the probe hybridizes to the target polynucleotide. This complementarity need not be perfect; there may be any number of base pair mismatches which will interfere with hybridization between the target polynucleotide and the probe polynucleotides. However, if the number of mismatches is so great that no hybridization can occur under even the least stringent of hybridization conditions, the sequence is not a complementary target sequence. Thus, by “substantially complementary” herein is meant that the probe hybridization domain and the hybridization domain in the target polynucleotide are sufficiently complementary to hybridize under normal hybridization conditions. The size of the probe polynucleotide may vary, as will be appreciated by those in the art, from 5 to 500 or more nucleotides in length, with probes of between 10 and 200 nucleotides being preferred, more preferably between 15 to 200, between 15 and 50 being particularly preferred, and from 10 to 35 nucleotides being especially preferred. The probe is preferably single-stranded.

The term “target polynucleotide,” “target” or grammatical equivalents herein means a polynucleotide, typically a naturally occurring nucleic acid, that is of interest to identify or quantitate in a test sample. The target polynucleotide may be all or a portion of a gene, a regulatory sequence, genomic DNA, cDNA, RNA including mRNA and rRNA, or others. The target polynucleotide may be from a sample, or a secondary target such as a product of a reaction such as a ligation product from an oligonucleotide ligation reader reaction, an amplification probe from oligonucleotide ligation amplification, product of an isothermal amplification, a PCR reaction product, etc.

The target polynucleotide has a hybridization domain that is substantially complementary to the hybridization domain of the probe polynucleotide. The hybridization domain of the probe and target polynucleotide may be any length, with the understanding that longer sequences are more specific. As will be appreciated by those in the art, the hybridization domain may take many forms. For example, it may be contained within a larger polynucleotide, i.e., all or part of a gene or mRNA, a restriction fragment of a plasmid or genomic DNA, among others. The probe polynucleotide is made to hybridize to the hybridization domain within the target polynucleotide to determine the presence or absence of the target polynucleotide in a sample. Accordingly, the region of the target polynucleotide that hybridizes to a region of a probe polynucleotide defines the hybridization domains for the probe and target.

The hybridization reactions outlined herein may be carried out in a variety of ways. For example, components of the hybridization reaction may be added simultaneously or sequentially. In addition, the reaction may include a number of other reagents such as salts, buffers, neutral proteins, e.g. albumin, detergents, etc., which may be used to facilitate optimal hybridization and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target polynucleotide.

In addition, double-stranded target polynucleotides are denatured to render them single-stranded so as to permit hybridization with the probe polynucleotides. A preferred embodiment utilizes a thermal step, generally by raising the temperature of the reaction to about 95° C., although pH changes and other techniques may also be used.

A test sample that may contain a target polynucleotide is contacted with a probe polynucleotide that is attached to the surface of a substrate to form an immobilized hybridization complex. A variety of hybridization conditions may be used, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target polynucleotide is present in excess, at Tm, 50% of the probes are all hybridized at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Hybridization conditions also include those disclosed by Sambrook et al., Molecular Cloning: A Laboratory Manual (2nd ed., Cold Spring Harbor Laboratory Press, New York), using a hybridization solution comprising: 5×SSC, 5× Denhardt's reagent, 1.0% SDS, 0.05% sodium pyrophosphate and up to 50% formamide. Hybridization can be carried out at 37-42° C. for six hours. Following hybridization, substrates can be washed as follows: (1) 5 minutes at room temperature in 2×SSC and 1% SDS; (2) 15 minutes at room temperature in 2×SSC and 0.1% SDS; (3) 30 minutes to 1 hour at 37° C. in 1×SSC and 1% SDS; (4) 2 hours at 42-65° C. in 1×SSC and 1% SDS, changing the solution every 30 minutes. The aforementioned incubation times may be reduced significantly.

Different stringent conditions can be used for hybridization. One exemplary formula for calculating the stringency conditions suitable for hybridization between nucleic acid molecules of a specified sequence homology (Sambrook et al., 1989):
Tₘ=81.5 C+16.6 Log [Na⁺]+0.41(% G+C)−0.63(% formamide)600/#bp in duplex

As an illustration of the above formula, using [Na⁺]=[0.368] and 50% formamide, with GC content of 42% and an average probe size of 200 bases, the Tₘ is 57° C. The Tₘ of a DNA duplex decreases by 1°-1.5° C. with every 1% decrease in homology. Thus, targets with greater than about 75% sequence identity might be observed using a hybridization temperature of 42° C. Such a sequence would be considered substantially homologous to the nucleic acid sequence of the present invention.

In another example, the hybridization conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer preferably is between 6.5 and 6.7. The wash buffer is 6×SSPET. 6×SSPET contains 0.9 M NaCl, 60 mM NaH₂PO₄, 6 mM EDTA, and 0.005% Triton X-100. Under more stringent acid array hybridization conditions, the wash buffer can contain 100 mM MES, 0.1 M [Na⁺], and 0.01% Tween 20. The aforementioned incubation times may be reduced significantly.

The hybridization is generally run under stringency conditions which allows formation of the hybridization complex only in the presence of target polynucleotide. Stringency can be controlled by altering a step parameter that is a thermodynamic variable, including, but not limited to, temperature, formamide concentration, salt concentration, chaotropic salt concentration, pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding.

In a preferred embodiment, the probe polynucleotides are designed for use in genetic diagnosis. For example, probes can be made to detect target polynucleotides such as the gene for nonpolyposis colon cancer, the BRCA1 breast cancer gene, P53, which is a gene associated with a variety of cancers, the Apo E4 gene that indicates a greater risk of Alzheimer's disease, allowing for easy presymptomatic screening of patients, mutations in the cystic fibrosis gene, or any of the others well known in the art.

Suitable target polynucleotides may also be associated with: (1) viruses, including but not limited to, orthomyxoviruses, (e.g. influenza virus), paramyxoviruses (e.g. respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picomaviruses, and the like; (2) bacteria, including but not limited to, a wide variety of pathogenic and non pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia, Y. pestis, Pseudomonas, e.g. P. aeruginosa, P. putida; Chlamydia, e.g. C. trachomatis; Bordetella, e.g. B. pertussis; Treponema, e.g. T. palladium; and the like, (3) yeasts, and (4) fungi such as Aspergillus.

When pathogens such as bacteria are being detected, the preferred target polynucleotides include rRNA, as is generally described in U.S. Pat. Nos. 4,851,330; 5,288,611; 5,723,597; 6,641,632; 5,738,987; 5,830,654; 5,763,163; 5,738,989; 5,738,988; 5,723,597; 5,714,324; 5,582,975; 5,747,252; 5,567,587; 5,558,990; 5,622,827; 5,514,551; 5,501,951; 5,656,427; 5.352.579; 5,683,870; 5,374,718; 5,292,874; 5,780,219; 5,030,557; and 5,541,308, all of which are expressly incorporated by reference.

The probe polynucleotide may be complementary to a target polynucleotide region in an organism's genome and thus allow the detection, quantitation, and analysis of the organism in a test sample. Different polynucleotide probes, which may be complementary to various regions in a specific organism's genome, may be attached to a single identifiable region. This may enhance sensitivity to detect a specific organism in a test sample.

If required, the target polynucleotide is prepared using known techniques. For example, the sample may be treated to lyse the cells, using known lysis buffers, electroporation, etc., with purification and/or amplification as needed, as will be appreciated by those in the art. The target polynucleotide may be amplified as required; suitable amplification techniques are outlined in PCT US99/01705, hereby expressly incorporated by reference. In addition, techniques to increase the amount or rate of hybridization can also be used; see for example WO 99/67425 and U.S. Ser. Nos. 09/440,371 and 60/171,981, all of which are hereby incorporated by reference.

In one embodiment, polynucleotides in the test sample are treated to produce smaller fragments, such as by sonication, hydrodynamic flow prior to hybridization or digestion with one or more restriction endonuclease. This treatment can reduce the length of target polynucleotides.

The substrates of the invention are used for attachment of probe polynucleotides to identifiable regions on the surface of the substrate. By “substrate” or “solid support” or other grammatical equivalents herein is meant any material that can be modified to contain discrete individual sites appropriate of the attachment of probe polynucleotides. Suitable substrates include glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), etc, polysaccharides, nylon or nitrocellulose, resins, silica or silica-based materials including silicon and modified silicon, carbon, metals, inorganic glasses and a variety of other polymers. The substrate may comprise planar chips, bead arrays, microarrays (Schena, M., Imicroarray Analysis (2003), John Wiley & Sons, Inc. Hoboken, New Jersey), membranes, microwell plates, encoded regions (e.g., encoded particles) (Braeckmans, K., et al., “Scanning the Code,” Modern Drug Discovery, February 2003, p. 28-32), three dimensional “gel pad” arrays, and those including electronic components (e.g. Nanogen).

The probe polynucleotide may be attached to the surface of substrates using photolithographic techniques (such as the Affymetrix GeneChip™), spotting techniques (e.g. Synteni and Incyte), printing techniques (Agilent and Rosetta).

As used herein, the term “attached” or grammatical equivalents refers to covalent as well as noncovalent attachment to describe when the attachment of a probe polynucleotide to a substrate. For example, a reactive functional group on a probe polynucleotide can react with another reactive group on the surface of the substrate to form a covalent linkage. An example of a probe polynucleotide having a free amino group is capable of forming a covalent bond with an aldehyde group on the surface of the substrate. Alternatively, a member of a binding pair can be immobilized on the surface of the substrate where the other member of the binding pair is attached to the probe polynucleotide. Upon application of the probe to the substrate, a noncovalent interaction occurs between the members of the binding pair. A well known example is streptavidin binding with biotin although other binding pairs can be used. Strong binding of the probe to the surface of the substrate may permit the use of probes in subsequent analysis.

As used herein, “identifiable region” refers to a region on the surface of a substrate that can be identified by way of x-y coordinates, e.g. on a planar surface or by the coordinates of micr6wells in a microwell plate. Alternatively, coded regions can be used so that the detection of the fluorescence waveform and be correlated with the code for the particular region (see Braeckmans, K. S., et al., “Scanning the Code,” Modern Drug Discovery, February 2003, p. 28-32). Identifiable regions may contain any concentration or density of probe polynucleotides. A preferred density is ˜2.6×10⁵ molecules/μm²˜2.6×105 molecules/mm2. See also Schena, M., Microarray Analysis, 2003, John Wiley & Sons, Inc., Hoboken, N.J.

The present invention finds particular utility in array formats, i.e. wherein there is a matrix of identifiable regions. By “array” herein is meant a plurality of probe polynucleotides in an array format; the size of the array will depend on the composition and end use of the array. Arrays containing from about 2 different probes to many thousands can be made. Generally, the array will comprise from two to as many as 100,000 or more, depending on the size of the substrates. Preferred ranges are from about 2 to about 10,000, with from about 5 to about 1000 being preferred, and from about 10 to about 100 being particularly preferred. In some embodiments, the probe polynucleotides may not be in array format; that is, for some embodiments, a single probe polynucleotide can be used to detect a target polynucleotide. In addition, in some arrays, multiple substrates may be used, either of different or identical compositions. Thus for example, large arrays may comprise a plurality of smaller substrates. For example, the array may comprise a bead array or a microplate. See, e.g., U.S. Pat. Nos. 5,591,578; 5,824,473; 5,705,348; 5,780,234 and 5,770,369; U.S. Ser. Nos. 08/873,598 08/911,589; WO 98/20162; WO98/12430; WO98/57158; WO 00/16089) WO99/57317; WO99/67425; WO00/24941; PCT US00/10903; WO00/38836; WO99/37819; WO99/57319 and PCTUS00/20476; and related materials, all of which are expressly incorporated by reference in their entirety.

The hybridization of this sample and probe polynucleotides may be carried out in an environment where the temperature is controlled. If double-stranded polynucleotide is present, it may be necessary to denature the sample by raising the temperature followed by equilibration at an appropriate temperature for carrying out the hybridization based on the G/C content of the hybridization domains and the components of the hybridization buffer. This may occur independently of the apparatus of the invention. In this case, the substrate may be transferred to a platform within the apparatus so that the regions of the substrate can be placed in optical communication with the fluorescence detection system.

The apparatus may also integrate sample preparation, purification, hybridization, signal detection, and data analysis. Crude samples (e.g., bacterial cells, crude bacterial cell lysate containing proteins, carbohydrates, lipids, DNA, RNA, etc.) can be treated appropriately (e.g., physical (heat) and chemical (NaOH)) prior to subsequent purification and/or hybridization. Samples that are not completely homogeneous may pass through a filtration system to retain large fragments (e.g., tissue or debris) to prevent obstruction.

In a preferred embodiment, thermocycler and thermoregulating systems such as controlled blocks or platforms are used in the apparatus of the invention to stabilize the temperature of the substrate to provide accurate temperature control for incubating samples from 0° C. to 100° C. This provides controlled hybridization conditions.

The apparatus of the invention may further comprise liquid handling components, including components for loading and unloading fluids at each region or set of regions. The liquid handling systems can include robotic systems comprising any number of components. In addition, any or all of the steps outlined herein may be automated; thus, for example, the systems may be completely or partially automated.

Fully robotic or microfluidic systems include automated liquid-, particle-, cell- and organism-handling systems including high throughput pipetting to perform all steps required for analysis. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations use cross-contamination-free liquid, particle, cell, and organism transfers. The system may perform automated replication of the test samples to regions of a substrate. This may include high-density transfers and serial dilutions.

Suitable staining methods for the present invention include, but are not limited to, pre-staining target and/or probe polynucleotides prior to hybridization, staining after the hybridization step without removal of the staining solution, or staining after the hybridization step followed by removal of the staining solution.

Suitable dyes for the present invention include any dye that has distinguishable fluorescence decay properties when noncovalently bound to a double-stranded polynucleotide, as opposed to when bound to a single-stranded polynucleotide. Such dyes may be derivatives of cyanine, indole, bisbenzimide, phenanthridine and acridine. Examples of appropriate dyes include those provided by Cosa et al., Chem. Commun. 689-690 (2000); Cosa et al., Photochemistry and Photobiology, 73(6):585-599 (2001); and Cosa et al., Analytical Chemistry, 74:6163-6169 (2002). Partially preferred dyes include SYBR Green I and PicoGreen (Molecular Probe Inc., Eugene Oreg.). The preferred dye should be environmentally sensitive and have very low fluorescence when the dye is free in solution. More preferably, the fluorescence decay should be insensitive to the polynucleotide sequence. Dye interactions with polynucleotides, may include but are not limited to, intercalation, groove binding (i.e. major or minor), and electrostatic interactions.

In a particularly preferred embodiment, SYBR Green I (possibly Dye 937 in U.S. Pat. No. 5,658,751) is used. SYBR Green I is inexpensive and has much lower mutagenicity than other staining dyes such as ethidium bromide (Singer, et al., Mutation Research. 439:37-47 (1999). Furthermore, ultra-sensitive detection (fluorescence intensity-based measurement) of SYBR Green I bound to DNA has been demonstrated with the detection of 80 fg of dsDNA (240 zmol of a 200 bp fragment) by capillary electrophoresis (Skeidsvoll and Ueland, Analytical Biochemistry, 231:359-365 (1995).

An intensity-based measurement to monitor hybridization could be used by introducing a calibration step. For example, this may involve, staining the probes before hybridization and then measuring the fluorescence. This fluorescence signal would be subtracted from the fluorescence signal measured after hybridization. However, such a procedure introduces extra steps and it is questionable whether one can consistently achieve and maintain the same degree of staining.

The invention preferably uses a fluorescence decay and/or lifetime measurement that allows a calibration-free reading that distinguishes the multiple contributions to the total fluorescence including, background fluorescence (autofluorescence), scatter, and the multiple components of the fluorophore whose spectra may be overlapping. Importantly, the fluorescence lifetime, which is an inherent molecular property, is resistant to affects of drift in light source intensity, wavelength dependence of detector response, light-scatter, and many other well-known factors that compromise the data in fluorescence intensity-based approaches.

After staining has occurred, a fluorescence decay measurement can be performed. Data from the fluorescence decay measurement can be analyzed in various ways to detect and quantitate hybridization. This may include, but is not limited to, calculating the fluorescence lifetime (s) and their relative contribution, from one or more identifiable regions, using a single-exponential analysis, multi-exponential analysis, or a global analysis. Polynucleotide hybridization may then be detected and quantitated by determining the relative contribution of the fluorescence lifetime component (s) associated with double-stranded polynucleotides as compared to the relative contribution of the fluorescence lifetime component(s) associated with single-stranded polynucleotides. Polynucleotide hybridization may also be detected by determining a difference between the fluorescence decay waveform collected after polynucleotides from a test sample were allowed to hybridize and a reference fluorescence decay waveform of the fluorophore bound to single-stranded polynucleotides. As an example, if no hybridization occurs, the fluorescence decay waveform may be the same as a known sample with all single-stranded polynucleotides. Alternatively, if a small amount hybridization occurs, the fluorescence decay waveform may be different. Polynucleotide hybridization may be quantitated by comparing the collected fluorescence decay waveform to the waveforms of samples with known degrees of hybridization.

In a preferred embodiment, the format is a bead, and the substrate is based on a bead array such as described in U.S. Pat. Nos. 6,288,220 and 6,391,562, US Patent Application Publication 20020132264, Kohara et al., Nucleic Acid Research, 30(16):e87 (2002), Kohara, Analytical Chemistry, 75(13):3079-3085 (2003); Noda et al., Analytical Chemistry, 75(13):3250-3255) (2003), all of which are incorporated herein by reference. In one specific example, a fluid sample containing denatured DNA (single-stranded DNA) is flowed in a reciprocal manner through a tube filled with a linear array of probe-labeled beads. This allows rapid hybridization (<10 min). The bead array is comprised of a capillary tube with an inside diameter slightly larger than the bead diameter. Beads with specific probes attached may be arranged in the capillary by a predetermined order.

In another preferred embodiment, the probes are arranged in respective spatially discrete areas on a substrate surface, like a traditional microarray slide. Each of these discrete areas have a predetermined or determinable position.

In another embodiment, the platform is a microwell plate, such as a 96-well plate. Furthermore, probes can be attached to an array of predetermined or determinable discrete areas on the substrate surface, e.g., within a single well.

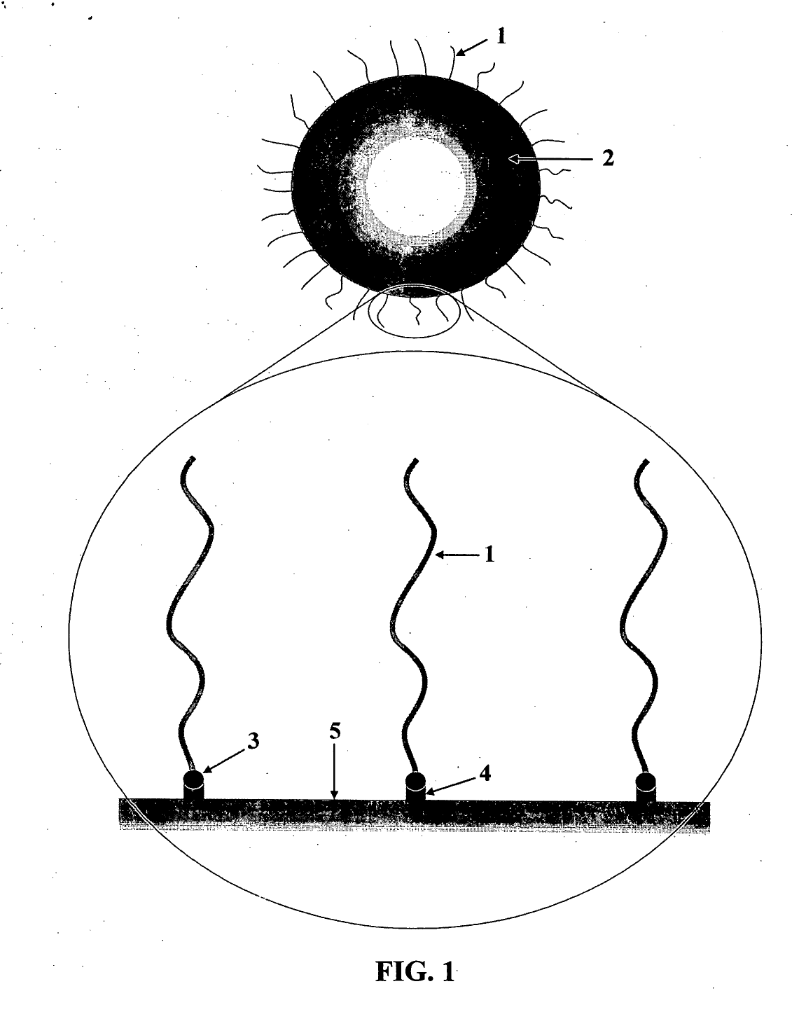

FIGS. 1-3 illustrate DNA hybridization using DNA probes attached to a substrate. FIG. 1 illustrates biotin 3 labeled probe ssDNA 1 (complementary to the target DNA), attached to a streptavidin 4 coated surface 5, which in this example is the surface of a glass bead 2.

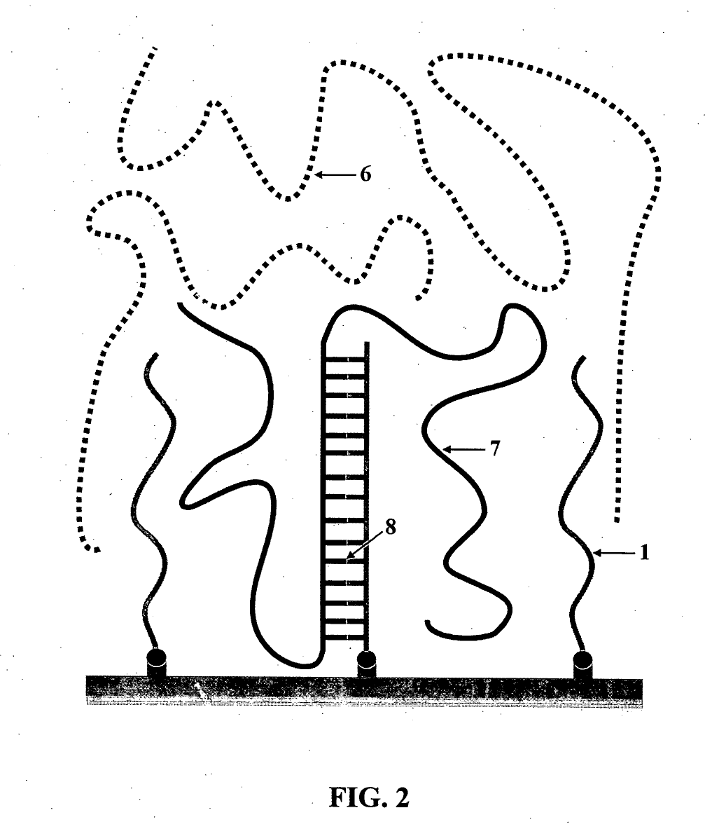

If a sample containing target DNA 7 and non-target DNA 6 is exposed to the probe 1 under suitable hybridization conditions, the complementary domain 8 of the target DNA will hybridize to the probe while the non-target DNA remains unbound, as schematically illustrated in FIG. 2. Following hybridization the non-target DNA may be removed.

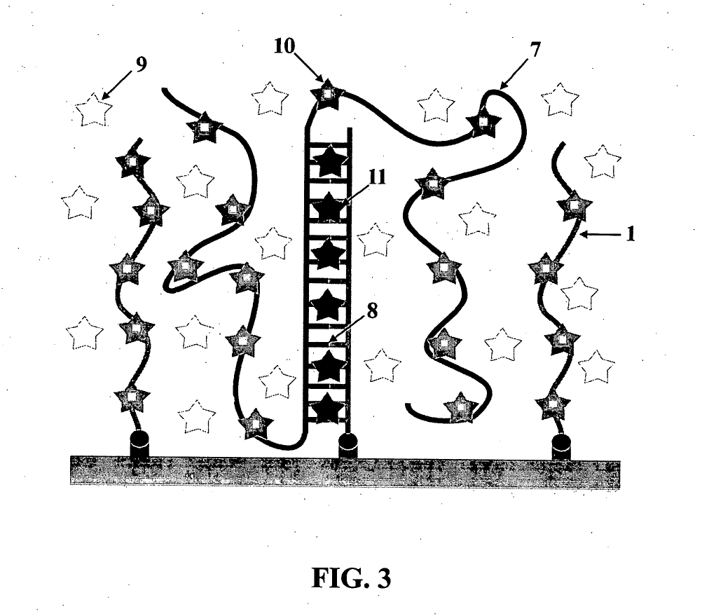

FIG. 3 illustrates target and probe DNA staining after hybridization without evacuation of the staining solution. The dye rapidly binds to both ssDNA and the hybridized dsDNA. The remaining unbound dye 9 in solution has little or no fluorescence. A suitable dye for the present invention will have distinguishable fluorescence decay properties when bound to a double-stranded polynucleotide 11, as opposed to when bound to a single-stranded polynucleotide 10.

Fluorescence Decay Detection System

Any fluorescence decay detection system or fluorescence decay measurement approach (e.g. frequency domain, time-correlated single photon counting, direct recording) can be used in the present invention.

In a preferred embodiment, the fluorescence decay detection system contains a pulsed light source and a digitizer. The detection system is designed to be in optical communication with the substrate when placed within the apparatus. Optical communication refers to the ability of the apparatus to sample fluorescent waveforms from one or more identifiable regions on the substrate and transmit them as an analog waveform to the digitizer. For example, optical communication between each of the identifiable regions and the detection system can be achieved: (1) by translating the substrate in two dimensions to position the identifiable region within the pulsed light beam, (2) translating the light and optics in two dimensions to sample the identifiable regions; and (3) scanning of the identifiable regions on the substrate. In a preferred embodiment, optical communication between each of the identifiable regions and the detection system can be achieved without performing a raster scan or generating an image of the regions.

“Pulsed Light Source”

The pulsed light source preferably produces pulses with short time interval of duration, e.g., in the sub-nanosecond or nanosecond, such as 0.4 ns to several nanosecond range. The pulsed light source may include, but is not limited to, a laser, laser diode (LD), or a light emitting diode (LED). In a preferred embodiment, the pulsed light source is a solid-state passively q-switched laser (“microlaser”).

“Digitizer”

The transient digitizer preferably can sample fluorescent signals at about a 0.5 gigahertz or higher sampling rate. A fluorescence decay waveform can be directly recorded following pulsed laser excitation. This allows rapid collection of fluorescence decay waveforms for processing data from many regions or samples. U.S. patent application Ser. No. 09/835,894 filed Jun. 20, 2003, corresponding to U.S. Patent Publication No. 2002/0158211, published Oct. 31, 2002; U.S. patent application Ser. No. 10/431,347, filed May 7, 2003, corresponding to U.S. Patent Publication No. 2004/0007675, published Jan. 15, 2004, each entitled “Multi-Dimensional Fluorescence Apparatus and Method for Rapid and Highly Sensitive Quantitative Analysis of Mixtures,” and U.S. patent application Ser. No. 10/600,319, filed Jun. 20, 2003, corresponding to U.S. Patent Publication No. 2004/0051656, published Mar. 18, 2004, entitled “System for Digitizing Transient Signals,” describe apparatus and methods to record fluorescence decay waveforms following pulsed laser excitation. Each of these applications are incorporated herein by reference. These methods are superior to tradition methods such as frequency domain or time correlated single photon counting in many aspects.

The conceptually simpler approach is to excite the fluorescence with a light pulse of short duration and to measure the temporal pattern of the subsequent fluorescence. The entire fluorescence decay curve can be measured following a single laser excitation pulse with a digital oscilloscope or transient digitizer, whose function is to track the output of a photomultiplier tube or other photodetector at closely-spaced time intervals. A plot of fluorescence intensity vs. time interval expressed relative to the time at which the excited state population is generated is commonly referred to as a fluorescence decay curve; a digitized representation of a transient signal as a function of time is also commonly referred to as a waveform or profile. In the ideal case that the time duration (pulse width) of the excitation pulse is much shorter than the fluorescence decay time, the lifetime can be determined from a plot of in Iₜ vs. t where Iₜ is fluorescence intensity at-time t relative to the laser pulse. Many mathematical deconvolution techniques are available for situations in which the excitation pulse duration is not infinitesimally short compared to the fluorescence lifetime. Deconvolution techniques require that the intensity be measured as a function of time for both the excitation pulse and the subsequent fluorescence pulse. Apart from a relatively uninteresting multiplicative factor, the mathematical relationship between the fluorescence and excitation waveforms involves a single parameter, namely the fluorescence lifetime. Each deconvolution procedure has the same goal, namely to determine the value of the lifetime that gives the best fit between the observed and predicted fluorescence decay curves.

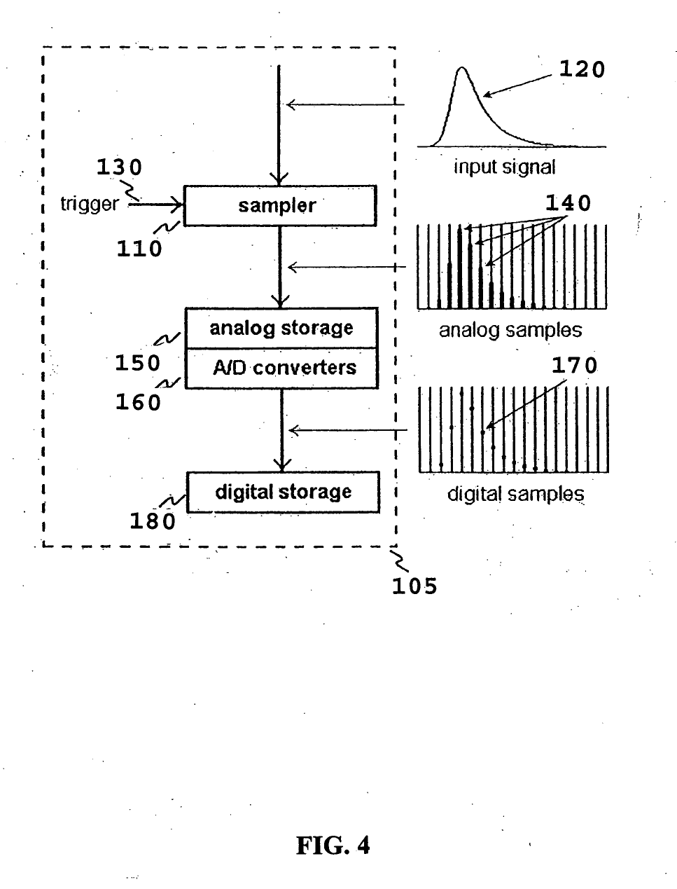

FIG. 4 illustrates digitizer 105 that includes a sampler 110 that samples time-dependent analog electrical signal 120. For one embodiment, a trigger signal 130 activates sampler 110. For another embodiment, sampler 110 generates one or more sampling strobes in response to receiving trigger signal 130. Each sampling strobe causes sampler 110 to obtain a sample 140 of signal 120 and store the sample 140 in analog memory (or storage) 150. Each sample 140 is a voltage or a charge that is proportional to signal 120. For some embodiments, analog memory 150 includes an array of memory elements (not shown), such as capacitors, that store a representation of time-dependent electrical signal 120 as a time-series of analog voltages or charges. Specifically, each element of the array stores a sample 140. For other embodiments, successive elements in the array correspond to a time increment no greater than 1 ns. For one embodiment, an A/D converter 160 is coupled to analog memory 150. A/D converter 160 operates on the analog data in analog memory 150 to generate the digital fluorescence decay waveform representation 170 that is stored in digital memory 180. For some embodiments, there is a single A/D converter for a single array, a single A/D/converter for each element of the array, etc.

In another embodiment, multiple input signals are received at digitizer 105. For this embodiment, each strobe causes sampler 110 to obtain a sample of each of the input signals and store the samples in analog memory 150. In one embodiment, analog memory 150 has a plurality of arrays each of which receives samples from a respective one of the input signals. There can be a single A/D converter for each of arrays or a single A/D converter for all of the arrays, etc. For one embodiment, the multiple input signals are copies of each other and are delayed in time relative to each other. For another embodiment, each of the multiple input signals are amplified or attenuated.

A design can also include a digital signal processor (DSP) that is useful to perform not only rapid processing of the digitized data that is the result of A to D conversions but also to provide intelligent control over one or more functions or parameters leading to output of the digitized data. In particular, a DSP can be made with CMOS or bi-CMOS technology and capacitor arrays of the kind that have been used to capture analog samples at high sampling rates can also be realized in CMOS or bi-CMOS. Thus, with CMOS or bi-CMOS (or any other chip-making methodology that permits realization of the essential components on a common substrate), it becomes possible to design a chip in which the DSP and the analog sample storage might be closely coordinated.

As used herein, DSP means any one of the conventional digital signal processor designs that has sufficient speed to handle the volume of data produced from A to D conversion within the time frames discussed further below. A DSP is typically characterized by optimization for numerical and vector processing, typically accomplished in part by having separate memories for data and for instructions. An example of a design of a commercially available DSP that is suitable for adoption in the present invention is the TMS320 family from Texas Instruments Incorporated. Specifically, a design such as the TMS320LF2812, might be adopted and adapted to eliminate the external bus, as part of integrating A to D conversion circuitry with the DSP. While only one DSP is depicted in the embodiments below, where greater processing power is needed, more than one could be used.

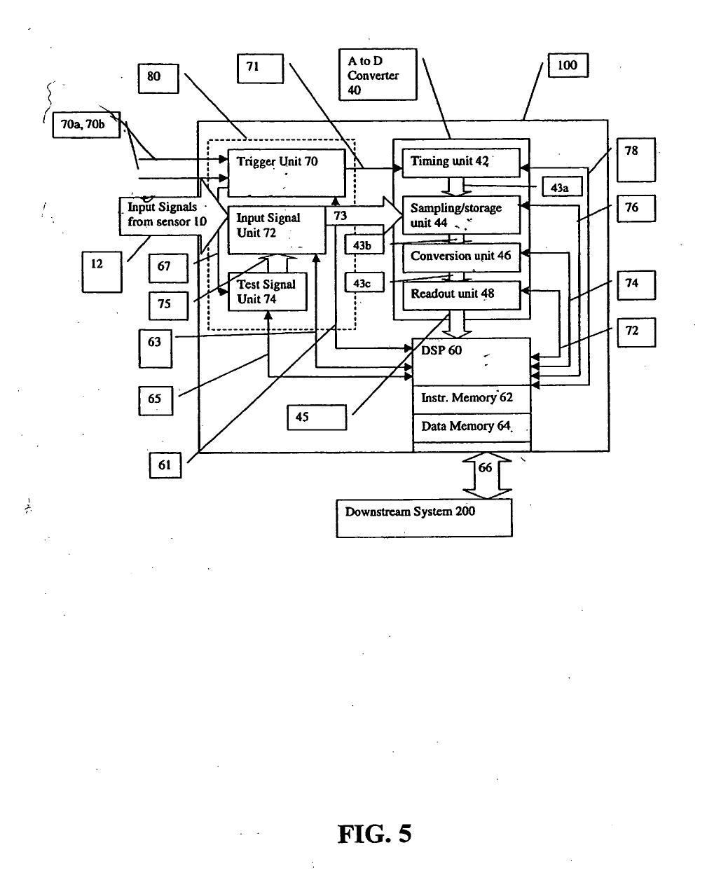

FIG. 5 shows the architecture of one embodiment of an integrated digitizer-DSP system 100. As seen in FIG. 5, the system has a DSP 60 with a separate data memory 62 and instruction memory 64 for control software and other software executed by the DSP. Output from the DSP 60 and from the system 100 occurs over a data link 66 to downstream system 200. Data link 66 may be a serial port to help keep the pin count for the output port low or, for some applications, may be a parallel port of the conventional kind.

A to D converter (ADC) 40 provides to the DSP on bus 45 the digital data that results from conversion of the analog inputs by ADC 40. The ADC 40 has a timing unit 42 that provides signals over internal bus 43a to a sampling and storage unit 44, which in turn provides the samples as outputs to conversion unit 46 over internal bus 43b. Sampling and storage unit 44 is in one embodiment a switch capacitor array with the capacity to accumulate charge in individual cells, which represent the samples having different analog levels that become digitized. Conversion unit 46 passes the now digitized data to a readout unit 48, using internal bus 43c. The DSP has communication paths 72, 74, 76 and 78 connecting it to the readout unit 48, the conversion unit 46, the sampling and storage unit 44 and the timing unit 42, respectively. Thus, the DSP has means for operably controlling a variety of parameters of operation of the ADC 40.

Also part of the digitizer system 100 are: a trigger unit 70, which receives external triggers from one or more trigger sources, e.g., 70a and 70b, and provides trigger signals over line 71 to timing unit 42; an input signal unit 72 that receives the analog input signals to be sampled from sensor 10, selects and conditions these signals in various ways and passes the resulting signals on to the sampling and storage unit 44 on communication path 73; and a test signal unit 74 that provides test signals to the input signal unit 72 via communication path 75. The DSP has communication paths 61, 63 and 65 connecting it to the trigger unit 71, the input signal unit 72 and the test signal unit 74, respectively, which together form a trigger/input module 80. (In an alternative embodiment the trigger/input module 80 includes only units 71 and 72.) The communication and control relationship of the DSP 60 to the various components is now described.

Trigger Unit 70

The trigger unit 70 is used to initiate the sampling that precedes an A to D conversion. (Although shown as integrated on chip 100, it is also possible for all or portions of trigger unit 70 to be implemented off-chip.) The timing of this sampling can be significant to applications. The trigger unit 70 has a variety of trigger facilities and parameters that are available for DSP control. The DSP 60 can enable or disable triggering, select the trigger source (e.g., select 70a or 70b), set the trigger gain, clear the triggered condition, set the trigger threshold level, and assert a trigger. The DSP can also set the time delay between the arrival of an external trigger and the triggering of the timing unit. Small changes in the delay can be used to implement equivalent time sampling (ETS) of repeatable input signals. Large changes in delay can be used to capture long transients as multiple segments or to move the sampling window to a region of interest. The trigger unit 70 can be held in a “ready” state without dissipating a lot of power (at least compared to a unit that is continuously clocked at a high rate), and it can “wake up” the rest of the system 100 (which could be in a low power state) when a trigger signal arrives.

To calibrate the trigger delay, the DSP 60 configures the trigger and test signal units 70, 74 so that a test signal is generated in response to the trigger signal. The DSP 60 can observe the effects of changes made by the DSP 60 to the trigger delay by inspecting the location of the test signal in the waveform read out from the ADC. Useful settings are saved by the DSP for later use.

Input Signal Unit 72

The input signal unit 72 may have one or more channels on which it receives the analog signals that are to be sampled. (Although shown as integrated on chip 100, it is also possible for all or portions of input signal unit 72 to be implemented off-chip.) The input signal unit 72 also has the ability to condition the incoming analog signals by adjusting the level with an offset, amplification or attenuation. The DSP 60 can select the input source, set offsets in input signal levels, and set gains.

To calibrate the offset, the DSP 60 sets the input signal unit to present a null signal and uses the ADC 40 to measure the result. The DSP can cause the input signal unit to change the offset or save the result and make a digital correction later.

To calibrate the gain, the DSP 60 controls the input signal unit to present DC signals with known levels. The DSP can also cause the test signal unit to generate signals with known amplitudes. The DSP uses the ADC output to observe changes made by the DSP to the gain. Useful gain settings can be saved by the DSP for later use.

If the same signal is available to more than one channel but with different delays, this DSP control provides a way to obtain interleaved samples. If the same signal is available to more than one channel but with different gains, this DSP control provides a way to extend dynamic range, as explained further below. [093] The DSP 60 may be able to detect an input out-of-range condition, by monitoring the input signal unit 72. If this event causes a condition flag to be set, the DSP 60 can read and clear this flag.

Test Signal Unit 74

Test signals are used to measure the trigger delay and the sampling rate. The signals used for measuring trigger delay are initiated by a signal from the Trigger Unit 70. The DSP 60 can adjust the timing and shape of the test signals. The DSP 60 enables and disables their use. Test Signal Unit 74 is also connected to Trigger Unit 70 via communication link 67. (Although shown as integrated on chip 100, it is also possible for all or portions of test signal unit 74 to be implemented off-chip.)

Timing Unit 42

The timing unit 42 generates the sampling strobes for the ADC 40. The rate at which these are generated is adjustable, which also influences the interval of time during which they are generated (sampling window). The DSP 60 can set the rate at which the strobes are generated and the length of time during which the storage cells track the input signal. The DSP 60 receives a signal from the timing unit 42 indicating when the sampling is done.

More specifically, the amount of time that a sampling capacitor tracks the input signal can be selectable, such as by the DSP 60. For example, it could track for N sampling periods where N is a pre-selected number, such as, 1, 2, 4, 8, or 16. This selection of the number of sampling periods is independent of the sampling rate and the width of the sampling window.

The DSP 60 can calibrate the sampling rate by causing the test signal unit 74 to generate a signal with features that are separated by a known period of time. An example of such a signal would be a clock signal. This signal is digitized by the ADC and the DSP uses the ADC output to determine the current sampling rate. The DSP then increases or decreases the sampling rate accordingly. As an alternative, a delay locked loop could be used to control the sampling rate. The DSP 60 could select the number of clock pulses from a clock and use this to define the width of the sampling window and thereby the sampling rate.

Sampling & Storage Unit 44

The sampling gates are essentially integrated into the storage unit; that is why the two functions, sampling and storage, are pictured as one unit. The DSP 60 can set the reference voltage level for the storage cells. The storage cells are organized as a matrix of capacitors, with multiple channels. The multiple cells in each channel are converted in parallel by presenting them in parallel to the conversion unit 46. The DSP 60 selects the channel to be presented to the conversion unit 46. There is a bank of buffers (not shown) between the storage cells and the A/D converters. These buffers are in one embodiment considered part of the sampling and storage unit 44. The DSP 60 can set the reference voltage level for these buffers. The DSP 60 can be programmed to set the voltage to which the capacitor cells are to be initialized or not to initialize the capacitors. In the latter case, the capacitors are “initialized” to their values from the previous sampling operation (subject to any leakage of charge during the interval between sampling operations).

Conversion Unit 46

The conversion unit uses a ramped reference voltage or an adjustable DC threshold to perform the determination of the analog level present in a cell. The DSP 60 can set the comparator reference voltage level, reset the ramp, start the ramp, control the ramp speed, start the counter for counting levels, advance the counter, set the range over which the counter will count, and reset the counter. The conversion unit 46 can send and the DSP 60 can receive a signal indicating that all the comparators have fired and/or a separate signal indicating that at least one comparator has fired. The DSP 60 can select between the ramp and the adjustable DC threshold. The DSP 60 can force the latches in the readout unit 48 to be loaded with the current counter output.

The DSP can measure and set (and thereby calibrate) the ramp speed by causing the input signal unit to present various DC levels to the ADC. The differences between the outputs of the ADC for the various levels are a measure of the ramp speed. The DSP can increase or decrease the ramp speed accordingly. The DSP may also control the duration of the time interval between the start of the ramp and the start of the counter.

Readout Unit 48

The readout unit 48 holds the digitized data in either serial or randomly addressable form in readiness for the DSP 60. The DSP 60 can shift out or select from this unit the data and permit the data to be driven onto the DSP data bus 66. If there is a known pattern of non-uniformity in the cells that have provided the digitized values, the DSP 60 can use a correction table, formula or other corrective reference and computation to apply corrections to deal with cell-to-cell variations. Cell-to-cell result variations are caused by differences in the circuit elements constituting the sampling cells (the switches and capacitors), the storage unit output buffers, and the comparators in the A/D converters. The DSP can measure these variations by using the output of the ADC when the input is a DC level. The DSP can set the DC level via its connections to the input signal unit. Dependence on various properties of the input signal (e.g., level and rate of change) can be measured by generating signals with the desired properties, which may involve coordination with the test signal unit. The results of these measurements are used by the DSP to apply corrections to acquired waveforms.

Output Port 66

The DSP 60 can communicate (exchange data with) an external device, such as a PC, using output port 66. Depending on the number of samples taken and any preprocessing that can be done by the DSP 60, the size of the sample record to be delivered from a digitizer chip 100 can vary. The digitizer becomes a more effective part of an overall digital sampling solution, to the extent it is programmed with instructions for preprocessing that remove unnecessary data or otherwise optimize the size of the sample record.

Power Levels

In many applications, power consumption is a significant variable, due to thermal considerations, limitations on available power, etc. The DSP 60 can use communication links to various elements in system 100 with which the DSP has communication, including those in the ADC 40 or within the DSP 60 itself, to reduce power usage by idling circuits within the system 100, reducing the frequency of their use, or using low power operational modes. Power conservation features can be of two types, depending on whether or not they prevent the digitizer from being able to respond to a trigger event; the latter enabling greater conservation but placing the digitizer in an inactive mode.

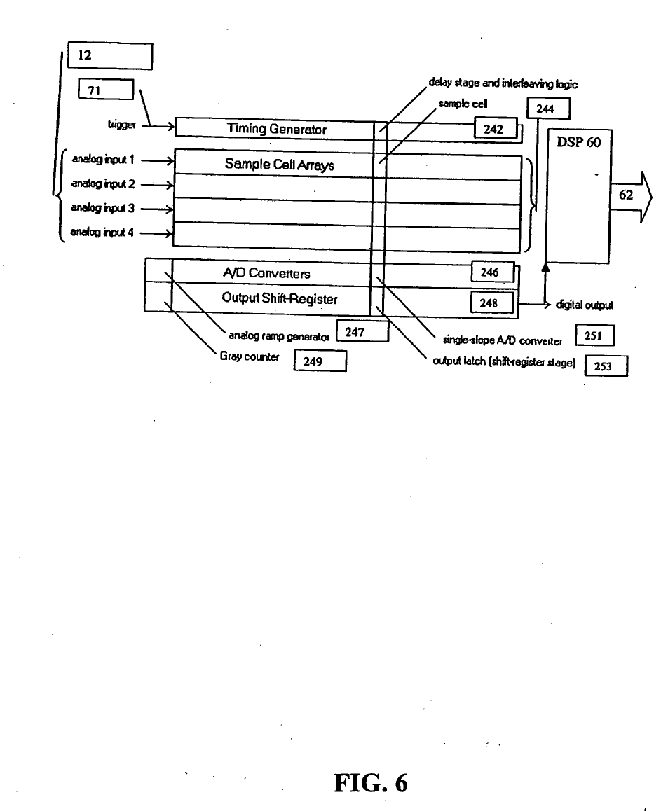

Turning now to FIG. 6, further details of the ADC 40 and its linkage to DSP 60 are discussed. The structure of portions of the ADC 40 is based on the analog transient waveform digitizer described in B. Greiman, et al., “Digital Optical Module & System Design for Km-Scale Neutrino Detector in Ice” Lawrence Berkeley National Laboratory, Jun. 20, 1998.

Timing Generator 242

The trigger signal (from trigger unit 70, see FIG. 5) received by the timing generator 242 initiates a timing signal from the timing generator 242 that propagates through the delay stages and interleaving logic, generating the strobe signals needed to control the sampling operations of the individual sample cells in the sample cell arrays 244. (FIG. 6 shows schematically one strobe path from the timing generator into a “column” in the sample cell arrays.) The sampling speed is determined by the propagation speed, which in turn is controlled by an input current bias. It is useful to note that this sampling speed is not governed by the clock speed of the DSP 60, and can be much faster. In one embodiment, the sampling speed is about 0.5-20 gigahertz, preferably about 1-10 gigahertz.

Another feature of one embodiment of the timing generator appears in the arrangement shown in FIG. 6, which is that the timing for the sampling comes from a “tapped delay line”, made from a sequence of delay stages. Sampling begins when a trigger 71 arrives at the timing generator. If the trigger is derived from the transient to be sampled (or whatever caused the transient), then it is synchronized with the transient and, because the triggering starts the sampling, the sampling is also synchronized with the transient. Consequently, if the transient is repeatable, and the system acquires the waveform multiple times, the samples of the different waveforms will all “line up” (in time). Or, if desired, the system can insert a small delay and “shift” the waveforms relative to each other so that a more detailed composite waveform can be constructed by combining multiple shifted waveforms. Most other samplers use a clock to determine when to sample. Sampling begins with the first clock event after the trigger event. The difference between these two events is random and introduces “jitter” into the position (in time) of each waveform. This makes it more difficult to combine waveforms. In the embodiment shown, such combining is facilitated.

Sample Cell Arrays 244

Analog samples of the input signals (from input signal unit 72, see FIG. 5) are held in the sample cells within the sample cell arrays 244. Each row of sample cells is a channel. In one embodiment, the number of sample cells in a row is about 50-2000, preferably about 128 or 1024. In the embodiment shown, during the sampling phase, analog samples of four signals are acquired concurrently and held in the cells of the four channels. (While in the one embodiment shown there are four channels, more or fewer channels, including just a single channel, are also possible.) The analog samples are passed to the A/D converters 246, one full channel at a time, during the conversion phase. One converter corresponds to each “column” in the sample cell arrays. The many columns mean that this is a highly parallel structure and suitable for integration on a chip. As an alternative, each channel has only one associated A/D converter, which operates with sufficient speed that it can perform serial conversion of all the analog samples within the required repetition interval.

A/D Converters 246

All the samples of a single channel are converted, in parallel, from analog to digital form by an array of single-slope A/D converters (one shown at 251). The A/D converters share the outputs from an analog ramp generator 247 and a Gray counter 249. External signals set the ramp speed, start and reset the ramp, and reset and advance the counter. The counter output is latched into individual output latches of a shift register stage 253, as comparators detect the ramp output passing by the voltage levels of the associated sample cells.

Readout Shift-Register 248

During the readout phase, the output latches are in one embodiment configured as a shift register. The latched values appear at the output of the readout-shift register.

Control by DSP

Operation of the ADC 40 and the trigger/input module 80 is variable based on a number of parameters. The DSP 60 gives the flexibility needed to quickly adapt the ADC's operation to various sampling and conversion methods that are found useful during the development of applications for the embodiments shown. The DSP 60 can also flexibly control operation of components of the trigger/input module 80. In either case, control may be based on signals from or states sensed within the ADC 40 and the trigger/input module. The DSP 60 can perform any of the following:
enable and disable triggering, select trigger sources, set the trigger threshold level, set the trigger delay, and generate a trigger signal
select input sources and condition the input signals by setting offsets and gains
adjust the timing and shape of the test signals and enable/disable them
set sampling and ramp speeds and optimize performance by setting the ADC bias currents and reference voltages
sequence the ADC control signals to step it through its sampling, conversion, and readout phases
convert and correct the digital data obtained from the ADC

Operating the ADC

The ADC 40 as shown in the embodiment of FIG. 5 has four channels and three operational phases: sampling, conversion, and readout. Sequencing of the ADC phases and channels is controlled by the DSP 60. The process starts with the sampling phase. Sampling begins when the ADC receives a ‘trigger’ signal. All four input channels are sampled concurrently. The DSP 60 waits until it sees the ‘trigger complete’ signal before it begins the conversion phase.

The DSP 60 starts the conversion process by selecting the channel to be converted, starting the analog ramp, and sending a clock signal to the Gray counter. The ramp speed and the counter clock frequency determine the step size. In one embodiment, the steps are of a size to permit 8-12 bits of resolution, preferably 10-12 bits of resolution and most preferably 10 bits. The ramp approach avoids the use of one comparator for each level of resolution, as is the case for “flash” A to D converters.

After conversion, the DSP 60 configures the output latches to form a shift register and reads out the digital values. To convert and read out the other channels, the DSP selects each one in turn and takes the ADC through the conversion and readout phases for the selected channel.

The DSP's ability to select a channel provides a facility for adjusting dynamic range. There are benefits when the amplitude of the input signal, as seen by the ADC 40, “matches” the input range of the ADC. It is a purpose of the input signal unit 72 to adjust the amplitude of the input signal to achieve this match. However, when the amplitude of the input signal is not known in advance (especially if it is a one-time signal), there may be no opportunity to make this adjustment. A solution to this problem is to route the signal to multiple input channels via paths in which there are amplifiers with differing gains. The input signal unit 72 can accomplish this function and generate multiple copies of the input signal, each copy having an amplitude differing from that of the other copies. For example, the copies may differ in scale by factors of 2. The ADC 40 samples all the copies at the same time, storing the analog samples for each copy in a separate array of storage cells. Now, for greatest efficiency, it is advantageous to convert and read out only the copy whose amplitude most closely matches the input range of the ADC.

What is needed, then, is a quick means by which the DSP 60 can identify the best copy without converting and reading out all the copies. One possibility is to check the input signal unit to see which signals (after amplification) exceeded the input range and pick the largest one that did not. The input signal unit 72 could perform this test and set flags for the DSP to sense. If this information is not available from the input signal unit 72, an alternative is to convert the smallest signal first and, based on the measured amplitude, select the best fit from among the remaining copies (if better than the smallest signal).

Another scheme is possible if the conversion unit 46 provides a DSP-readable indicator that at least one of the comparators has fired. In this case the DSP 60 can select a threshold against which the samples are to be compared and then test all the samples of one channel in parallel against this threshold. If at least one of the comparators fires, then the copy is too large. The DSP can use this capability to quickly find the largest copy that is not too large and take it through the conversion and read out processes.

Data Conversion and Correction

The data from the ADC is in a Gray code format. Before the DSP can perform arithmetic operations with this data, it must be converted to binary code format. This conversion can be done by hardware during readout. The DSP can correct for fixed sample-to-sample variations that are seen when a null input signal is digitized. Measurements of these variations, called pedestals, can be stored in the DSP and subtracted from the data after Gray-to-binary conversion. Each channel has its own set of measured pedestals.

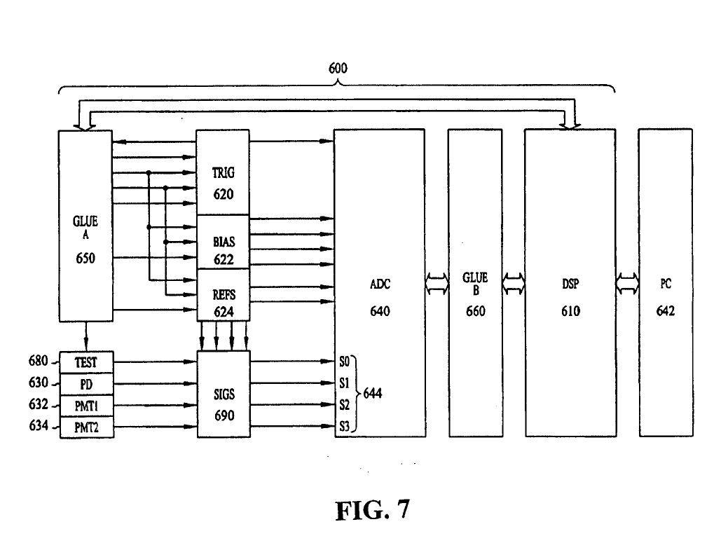

FIG. 7 shows a further embodiment of a digitizer 600 for providing digitized data from an optically detected event to a PC, which is now described. The functions of the embodiment are realized in hardware, software, or a combination of the two. The software components reside in the program storage of the digital signal processor (DSP) 610. The hardware components are pictured in the block diagram of FIG. 7. The ADC 640 and DSP 610 are as described above. The other hardware components in FIG. 7 are described below.

DSP Control of the Trigger, Bias Currents, and Reference Voltages (GLUE A 650): For precise and repeatable control, digital to analog converters (DACs) are built into the TRIG 620, BIAS 622, and REFS 624 components. These DACs control the trigger reference level, the sampling speed and ramp speed bias currents, a number of reference voltages (including the PD 630, PMT1 632, and PMT2 634 signal offsets), and the TEST 680 signal offset. The DACs are programmed by the DSP. Changes may be made from the PC 642 by sending commands to the DSP.

Signal Sources (TEST 680, PD 630, PMT1 632, PMT2 634): The ADC has four input channels (S0-S3) 644. In this example, one channel, the TEST channel, is used for DSP-generated patterns. Another channel, the PD channel, accepts signals from a PIN photodiode. A transimpedance amplifier (TIA) (not shown) may be inserted between the photodiode and the ADC to keep the bias voltage constant, provide some gain, and drive the ADC input. The other two channels, PMT1 and PMT2, accept signals conducted by a 50-ohm coaxial cable. A typical use for one of these channels is to connect to a photomultiplier tube (PMT).

Triggering (TRIG 620): A reverse-biased PIN photodiode is used to detect the laser pulse. A comparator generates the trigger signal when the output of the photodiode exceeds a reference level. The trigger signal must remain active while the ADC is sampling, so a means of latching the signal is needed. The DSP clears the latch when the digitizer is ready to receive the next trigger.

Bias Currents and Reference Voltages (BIAS 622 & REFs 624): There are a number of bias currents and reference voltages that must be set within certain ranges for proper operation of the ADC and the analog input circuitry. Some of these may be variable and others may be set to fixed nominal values. Two useful variable settings are the current biases that control the sampling speed and the ramp speed. These determine the time and amplitude resolutions by which waveforms are sampled and digitized.

Input Signal Conditioning (SIGS 690): The input signals may be AC- or DC-coupled and may have a DC offset added. After this, the TEST, PMT1, and PMT2 channels have an amplifier with a fixed or variable gain. The offsets and gains may be adjustable by the DSP. There may also be input protection circuitry. Out-of-range inputs could be reported to the DSP.

The DSP-ADC Interface (GLUE B 660): The control and status pins of the ADC may be connected to individually programmable digital I/O pins of the DSP. Use is also made of the DSP's data bus. During the readout phase, the digitized data from the ADC is driven onto the bus and loaded into RAM within the DSP. The glue logic includes the tri-state drivers and control logic to perform this read operation.

Timing for Sampling and Digitizing

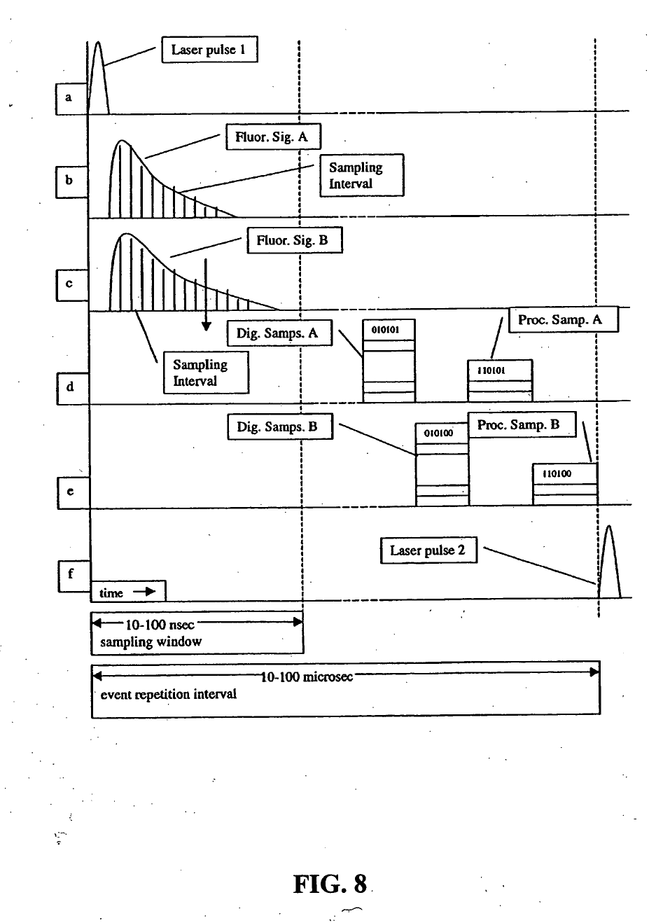

One or more hybridization complexes with a fluorophore are induced to fluoresce by one or more laser pulses. The first pulse is shown at line a of FIG. 8 and the second (next consecutive) pulse is shown at line f. The interval between pulses may be called the repetition frequency interval. The present design contemplates event rates of greater than 10 kiloHz, but remaining significantly (factor of 10 to 100) below the sampling rate. Multiple pulses can be used if there is a scarcity of fluorescence emissions so that it is necessary to have repeated observations in order to build up the points of a waveform representing the fluorescence . The digitizer may also be used to examine multiple samples, each of which is subjected to one or more laser pulses. The desire to increase throughput requires that laser pulses be spaced with a time interval that minimizes the delay until the next sample.

Each laser pulse will have a relatively short time interval of duration (in the sub-nanosecond, in one embodiment about 0.4, to several nanosecond range) and each corresponding fluorescence waveform will be somewhat longer but also in the several nanosecond range. In order to get a good waveform of the fluorescence emission, it is desirable to take analog samples at a 1 to 4 gigahertz rate. Thus, in one embodiment the sample rate interval for one sample is approximately 1/10⁹ second. The duration of an entire sampling window is on the order of about 10 to 100 nanoseconds. By contrast, the duration of the event repetition interval is about 10 to 100 microseconds. Thus, because sampling occurs at the start of the event repetition interval, almost all of this latter period is available for processing the analog samples, which are collected in the first 10 to 100 nanoseconds. FIG. 8 shows that the Digitized Samples and any Processed Samples appear late in the total interval between laser pulses. (Note that the length of the sampling window and event repetition interval are not shown to scale in FIG. 8; the event repetition interval is much foreshortened and Processed Samples A and B would typically be more staggered in time.)

In FIG. 8, there are two fluorescence signals that are observed following the laser pulse shown on line a. The two waveforms of the two observations appear on lines b and c of FIG. 8. Each analog sample value is depicted by a vertical line under the curve. (There can be one or more of such waveforms, which may be, e.g., observations at different frequencies or have a polarization difference relative to one another. The digitizer as depicted in FIG. 6 is configured to handle up to four waveforms captured during a sampling window.) Once each of the waveforms has been digitized, digital sample values can be stored in digital memory, such as a bank of registers. A sequence of such values is schematically shown as a column of binary numbers labeled “Digitized Samples” at lines d and e of FIG. 8.

DSP 60 may have control software for performing an additional level of processing on the raw digital sample values. Preferably, the processing reduces the size of the data record to be outputted, but it may also add additional measures derived from the raw sample data, such as waveform summing. This will result in another set of data or processed record, shown as a shorter column of binary numbers labeled “Processed Samples” at lines d and e of FIG. 8. DSP processing may reduce record size and alleviate output timing problems from the DSP. Some data calculated by the DSP can be control data, such as “good sample complete” flags to be used as part of a control loop on the chip or including the chip and the outside system, such as a microwell plate reader, or other means by which the digitizer obtains signals corresponding to a different sample. A control loop might be used to move a sample, move a laser-sensor assembly or to change the optical path between the two, as with a movable mirror.

DSP Functions

Placement of the DSP on the chip leads to the usual advantages of speeding inter-component communication, but there are other advantages that arise when DSP-executed functions can occur on chip. A particular benefit is reduced power consumption. This can be particularly useful in applications where a digitizer is needed at a point of signal origination. The present design permits embedding the digitizer/DSP at a point of signal origination, such as a particular location in a transmission network or circuit, even when that point has little power available or limited thermal requirements. This embedded digitizer/DSP also permits real time, digitized data to be generated without bringing in a large piece of equipment. A further benefit of the ADC and DSP integrated on one chip is that while there are internal paths with many lines (particularly where parallelism is used), there are fewer pins or contact points for external signals. This latter also helps reduce overall chip size.

It should be understood that the above-described embodiments and the following examples are given by way of illustration, not limitation. Various changes and modifications within the scope of the present invention will become apparent to those skilled in the art from the present description.

### EXAMPLE

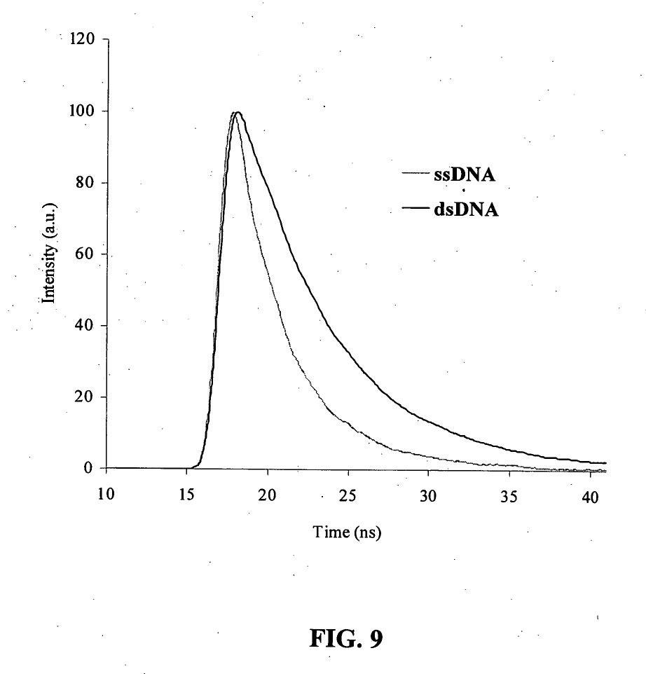

FIG. 9 shows fluorescence decay curves collected by directly recording the waveform following a laser excitation pulse. The significant difference in the fluorescence decay waveforms of two samples, one containing a solution of SYBR Green I bound to ssDNA and the other containing a solution of SYBR Green I bound to dsDNA, is evident. The ssDNA sample was a 20 bp probe oligonucleotide (sense strand) free in solution. The dsDNA sample was composed of a 20 bp fragment developed by annealing the probe oligonucleotide to and complementary target oligonucleotide (antisense strand). If the data are interpreted in terms of single exponential decay, the lifetimes are 2.84 ns and 5.33 ns for ssDNA and dsDNA, respectively. Multi-exponential decay model can also be used for analyzing the fluorescence lifetime data.

A change in the fluorescence decay waveform shape from that representative of a sample containing all ssDNA, signifies an increase in the amount of dsDNA or DNA hybridization. The data can be processed in many different ways. For example, a 3-component global analysis of the decay curves can be performed, but a much simpler method is just to compare the shapes of the fluorescence decay curves. The shape comparison introduces a single scaling parameter that normalizes for intensity differences. The figure of merit is the sum of squares of the residuals, commonly referred to as chi-squared. The chi-square can be either weighted or unweighted. In one example, the shape of two waveforms is compared by computing the degree of overlap (or equivalently, the degree of non-overlap) of the normalized waveforms. For instance, if Waveform 1 is represented as Vᵢ(t) and Waveform 2 is represented as Vⱼ(t), one can compute the difference function Dⱼᵢ(t)=Vⱼ(t)−alpha x Vᵢ(t). Chi-squared is the sum of the squares of the differences: Chi²=sum[Dⱼᵢ(t)²]. Then the value of alpha is varied to minimize the value of Chi². The smaller the value of Chi², the better the overlap of the two waveforms and therefore the more similar the two curves are.

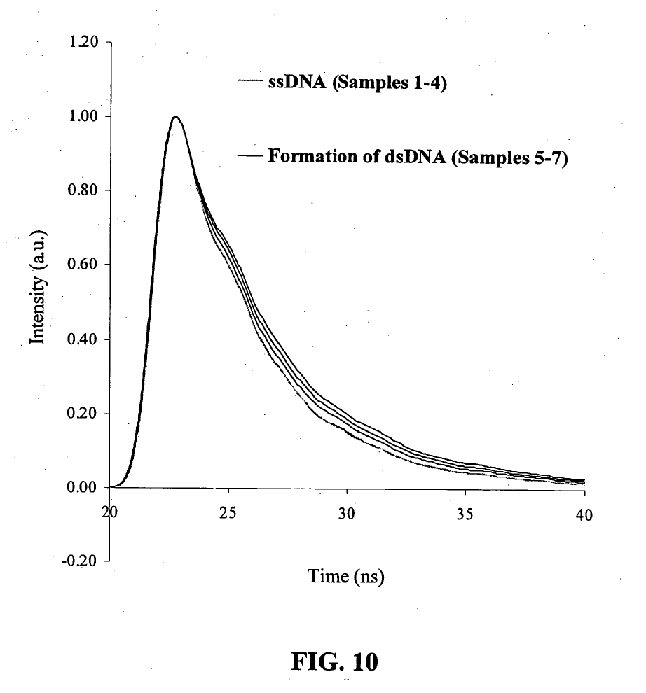

FIG. 10 demonstrates the ability to detect a very small degree of hybridization (measurements taken in cuvettes). This started with three samples that contained 1 nmol of probe ssDNA (20 bp, sense strand, oligonucleotide free in solution) in a 2 ml volume. To these samples, different amounts of the same probe ssDNA (20, 40, or 60 pmol) were added. In a parallel experiment, four samples that contained 1 nmol of probe ssDNA (20 bp, sense strand, oligonucleotide free in solution) were added with different amounts (0, 20, 40, or 60 pmol) of complementary target ssDNA (20 bp, antisense strand, oligonucleotide free in solution). All of the samples were treated with a hybridization step (ramped to 94° C. and then slowly cooled to 25° C.) followed by a measurement of the fluorescence decay curve from each sample (FIG. 10). Samples 1-4 contained varying amount of ssDNA but no dsDNA. Samples 5-7 contained increasing amounts of dsDNA (2% increments) relative to ssDNA.

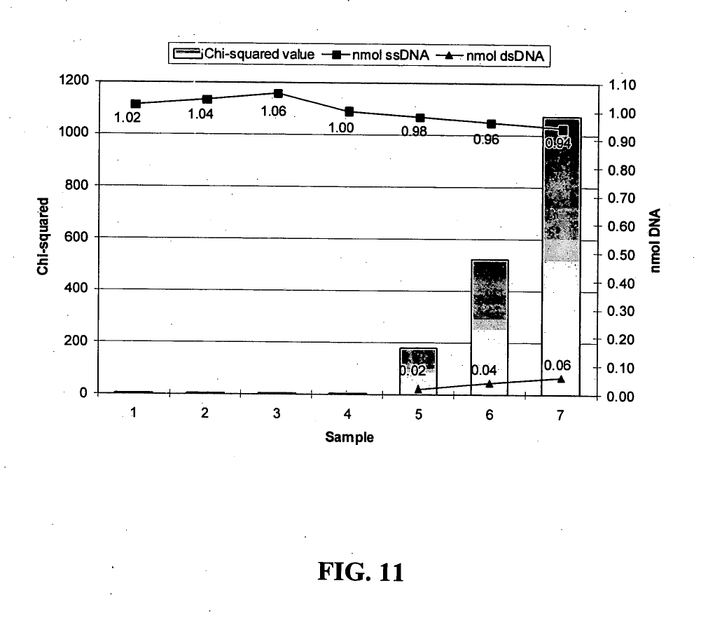

As shown in FIG. 11, the waveform changes are inconsequential as the ssDNA concentration is changed (samples 1-4). But when a small amount of dsDNA is formed by hybridization, the chi-squared value increases significantly (samples 5-7).

All of the above described fluorescence decay curve analyses can be performed automatically using computers or other processor-based systems.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. An apparatus comprising:
a fluorescence decay detection system comprising a pulsed-light source and a digitizer; and
a substrate comprising a plurality of identifiable regions each comprising a surface wherein at least one of said identifiable regions comprises a probe polynucleotide attached to said surface;
wherein said fluorescence decay detection system is capable of being in optical communication with each of said regions and of measuring the fluorescence decay or lifetime of a fluorophore.

2. An apparatus comprising:
a fluorescence decay detection system comprising a pulsed-light source and a digitizer; and
a substrate comprising a plurality of identifiable regions each comprising a surface wherein at least one of said identifiable regions comprises a probe polynucleotide attached to said surface;
wherein said digitizer comprises:
an array of memory elements that stores a representation of a time-dependent electrical signal corresponding to an analog fluorescence waveform signal from at least one of said identifiable regions as a time-series of analog voltages or charges; and
at least one analog-to-digital converter that transforms the time-series of analog voltages or charges into a corresponding digitized fluorescence waveform; and
wherein said fluorescence decay detection system is capable of being in optical communication with each of said regions and of measuring the fluorescence decay or lifetime of a fluorophore.

3. An apparatus comprising:
a fluorescence decay detection system comprising a pulsed-light source and a digitizer;
a substrate comprising a plurality of identifiable regions each comprising a surface wherein at least one of said identifiable regions comprises a probe polynucleotide attached to said surface;
wherein said digitizer comprises:
an array of memory elements that stores a representation of a time-dependent electrical signal, corresponding to an analog fluorescence waveform signal from at least one of said identifiable regions, as a time-series of analog voltages or charges;
at least one analog to digital converter that transforms the time-series of analog voltages or charges into a corresponding digitized fluorescence waveform;
at least one digital signal processor for operably controlling parameters of the digitizer and for receiving the digitized fluorescence waveform; and
wherein said fluorescence decay detection system is capable of being in optical communication with each of said regions and of measuring the fluorescence decay or lifetime of a fluorophore.

4. The apparatus of claims 2 or 3, wherein the analog to digital converter is configured with multiple converters to act in parallel on the time-series of analog voltages or charges in a memory to produce a corresponding digitized fluorescence waveform.

5 The apparatus of claims 1-3 wherein one or more of said regions comprise the same or different probe polynucleotides attached to said one or more regions.

6. The apparatus of claims 1-3 wherein said probe polynucleotides are covalently attached to the surface of said substrate.

7. The apparatus of claims 1-3 wherein said substrate configuration is selected from the group consisting of bead arrays, microarrays, membranes, microwell plates and encoded particles.

8. The apparatus of claims 1-3 further comprising a fluorophore present in at least one of said identifiable regions, wherein fluorescence decay of the fluorophore when noncovalently associated with a double-stranded polynucleotide is different from the fluorescence decay of the fluorophore when it is noncovalently associated with a single-stranded polynucleotide.

9. The apparatus according to claims 1-3, wherein said pulsed-light source comprises a laser or microlaser.

10. The apparatus according to claim 9, wherein said microlaser comprises a solid-state passively q-switched laser.

11. The apparatus according to claim 1-3, wherein said pulsed-light source comprises a light emitting diode (LED).

12. The apparatus according to claims 1-3 wherein said pulsed-light source comprises a laser diode (LD).

13. A method comprising:
forming a fluorescently labeled double-stranded polynucleotide hybridization complex comprising a probe polynucleotide attached to an identifiable region of a substrate, a target polynucleotide, if present in a test sample wherein said target polynucleotide has a hybridization domain substantially complementary to said probe polynucleotide, and a fluorophore that noncovalently interacts with a double-stranded polynucleotide, wherein the fluorescence decay or lifetime of said fluorophore when associated with a double-stranded polynucleotide complex is different from the fluorescence decay or lifetime of said fluorophore when associated with a single-stranded polynucleotide; and
measuring the fluorescence decay and/or lifetime of the fluorophore at said identifiable region, wherein the fluorescence decay and/or lifetime provides an indication of the presence or absence of said target polynucleotide in said test sample.

14. A method comprising
contacting a test sample with one or more identifiable regions on a substrate, wherein one or more of said probe polynucleotides are attached to the surface of said substrate and are substantially complementary to a hybridization domain in one or more target polynucleotides that may be present in said test sample, to allow formation of one or more hybridization complexes between said probe polynucleotides and said target polynucleotides;
contacting a fluorophore with said probe polynucleotide, said target polynucleotide or said hybridization complex, wherein the fluorescence decay or lifetime of said fluorophore noncovalently associated with a double-stranded polynucleotide is different from the fluorescence decay of said fluorophore noncovalently associated with a single-stranded polynucleotide; and
measuring the fluorescence decay and/or lifetime of the fluorophore at said one or more identifiable regions, wherein the fluorescence decay and/or lifetime determined for each of said one or more regions provides an indication of the presence or absence of said one or more target polynucleotides in said test sample.

15. The method of claim 13 or 14 wherein said fluorophore comprises derivatives of cyanine, indole, bisbenzimide, phenanthridine, and acridine.

16. The method of claim 13 or 14 wherein said fluorophore is SYBR Green I or Picogreen.

17. The method of claim 13 or 14 wherein said measuring comprises calculating the fluorescence lifetime(s) and their relative contribution from each of said one or more identifiable regions, using a single-exponential analysis, multi-exponential analysis, or global analysis, wherein formation of said polynucleotide hybridization complex is detected and quantitated by determining the relative contribution of the fluorescence lifetime component(s) associated with a double-stranded polynucleotide as compared to the relative contribution of the fluorescence lifetime component(s) associated with a single-stranded polynucleotide.

18. A method of claim 13 or 14 wherein formation of said polynucleotide hybridization complex is detected by determining the difference between a collected test fluorescence decay waveform and a reference fluorescence decay waveform of the fluorophore bound to single-stranded polynucleotides.

19. A method of claim 13 or 14 wherein the formation of said polynucleotide hybridization complex is quantitated by comparing a collected test fluorescence decay waveform to the waveforms of samples with known degrees of hybridization.

20. A method of claim 13 or 14 wherein a multiplicity of probe polynucleotides are used to detect a multiplicity of target polynucleotides in said test sample.

21. The method of claim 13 or 14 wherein one or more of said regions comprise the same or different probe polynucleotides attached to said one or more regions.

22. The method of claim 13 or 14 wherein aid measuring is with the apparatus of claims 1-3.
